(19) United States
(12) Patent Application Publication
(10) Pub. No.: US 2018/0098865 A1
(43) Pub. Date: Apr. 12, 2018

(54) PROSTHETIC SOCKET FIT SENSOR

(71) Applicants: Leticia Danira Miranda Mojica, El Paso, TX (US); Samuel Terrazas Quezada, El Paso, TX (US); Meagan Renee Kendall, El Paso, TX (US)

(72) Inventors: Leticia Danira Miranda Mojica, El Paso, TX (US); Samuel Terrazas Quezada, El Paso, TX (US); Meagan Renee Kendall, El Paso, TX (US)

(73) Assignee: THE BOARD OF REGENTS OF THE UNIVERSITY OF TEXAS SYSTEM, Austin, TX (US)

(21) Appl. No.: 15/727,191

(22) Filed: Oct. 6, 2017

Related U.S. Application Data
(60) Provisional application No. 62/404,847, filed on Oct. 6, 2016.

(51) Int. Cl. A61F 2/80; A61B 5/00; A61F 2/78

(52) U.S. Cl. CPC A61F 2/80; A61F 2/7812; A61B 5/4851

(57) ABSTRACT

An apparatus for achieving a best possible prosthetic fit in a prosthesis system for an amputee includes a sensor array embedded in a gel like substance slipped over a residual limb in a socket configuration with respect to a prosthesis system. Such an apparatus can also include data collection and transmission mechanism such as a microcontroller that communicates with the sensor array and which data from the sensor array to a computing device for processing and interpretation of the data in order to statically and dynamically assess a quality of fit of the prosthesis system for the amputee.

### CROSS REFERENCE TO RELATED APPLICATIONS

This application claims priority to U.S. Provisional Application No. 62/404,847 filed Oct. 6, 2016, which is incorporated herein by reference in its entirety.

### STATEMENT REGARDING FEDERALLY FUNDED RESEARCH

None.

### BACKGROUND OF THE INVENTION

### A. Field of the Invention

Embodiments are related to prosthetic devices for amputees. Embodiments are also related to devices for fitting prosthetic devices for amputees and for tracking the activity/use of the amputee's prosthetic device.

### B. Description of Related Art

Comfort, largely impacted by residual limb health, is a key indicator of prosthetic limb acceptance and use by amputees. Several factors can determine the comfort of a prosthetic limb, such as, for example, pressure distribution, level of humidity, temperature, and prosthetic alignment. While some tools exist that allow for quantitatively measuring these factors individually in a research environment, a tool does not currently exist, which measures multiple comfort indicators in a clinical setting.

Typically, prosthetists rely on their patients' feedback for an indication of how comfortable a socket is. However, discomfort levels for patients are subjective. Further, certain patients, such as diabetics, actually lose sensation in their residual limb and therefore do not know when their limb is actually in pain and at risk of injury. This limits their ability to report comfort and fit issues to their clinician. In these cases, a clinician's recourse is to rely on their prior experience and ability to “know” if there is a problem. Therefore, an inexperienced clinician may struggle to properly fit a socket properly for a patient who cannot adequately verbalize his or her comfort level.

### SUMMARY OF THE INVENTION

Certain embodiments are directed to solving various problems associated with fitting a prostheses by providing for an apparatus that assesses fit during the process of fitting a prosthetic socket based. In other aspects the methods and devices described herein can provide a history of use and information related to the current fit of the residual limb to clinicians.

It is yet another aspect of the disclosed embodiments to provide for an apparatus and system for assessing quality of fit of a prosthetic system for an amputee both statically and dynamically. In certain aspects a sleeve or sock device includes a sensor array. In particular instances the sensor array is embedded in a gel like substance. In certain aspects the gel-like substance is silicone or urethane. The apparatus is designed to be slipped over or receive a residual limb.

An apparatus for achieving a best possible prosthetic fit in a prosthesis system for an amputee includes a sensor array embedded in a gel like substance slipped over a residual limb in a socket configuration with respect to a prosthesis system. Such an apparatus can also include a data collection and transmission mechanism such as a microcontroller that communicates with the sensor array and transmits data from the sensor array to a computing device for processing and interpretation of the data in order to statically and dynamically assess a quality of fit of the prosthesis system for the amputee.

The sensor array can be implemented as a reusable sensor array that provides feedback regarding socket comfort and fit to clinicians by measuring some combination of the following: pressure distribution inside the socket, humidity level, temperature, and alignment of the prosthetic system and other comfort indicators. Such an apparatus is capable of assessing quality of fit statically (e.g., when the user is standing still) and dynamically (e.g., when the user is in motion). The apparatus can be configured in a variety of sizes for each limb amputation level and implemented in clinical, research, and home use. The apparatus is easy to clean and data can be collected and transmitted wirelessly to a computer for processing. The apparatus can work independently or in conjunction with software on a computer or smartphone or other computing device.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa.

The use of the word “a” or “an” when used in conjunction with the term “comprising” in the claims and/or the specification may mean “one,” but it is also consistent with the meaning of “one or more,” “at least one,” and “one or more than one.”

The term “about” or “approximately” are defined as being close to as understood by one of ordinary skill in the art. In one non-limiting embodiment the terms are defined to be within 10%, preferably within 5%, more preferably within 1%, and most preferably within 0.5%.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods of making and using the same of the present invention can “comprise,” “consist essentially of,” or “consist of” particular ingredients, components, blends, method steps, etc., disclosed throughout the specification.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1 illustrates a schematic diagram of an apparatus for facilitating the best possible prosthetic fit in a prosthesis system for an amputee, in accordance with an example embodiment.

FIG. 2 illustrates an example of a system for facilitating the best possible prosthetic fit in a prosthesis system for an amputee, in accordance with an example embodiment.

FIG. 3 illustrates an example of a system for facilitating the best possible prosthetic fit in a prosthesis system for an amputee, in accordance with another example embodiment.

### DETAILED DESCRIPTION OF THE INVENTION

Individuals who have suffered an amputation and seek to restore their mobility are often fitted with an artificial or prosthetic limb to replace the functionality of the amputated limb. One of the key components of this device is the socket, which is the interface between the residual limb and the artificial limb. In prosthetics, an amputee normally dons a prosthetic device by inserting his/her residual limb into a socket portion of the prosthesis. It is now fairly typical for an amputee to first place a prosthetic liner over the residual limb prior to insertion of the residual limb into the prosthetic socket, the prosthetic liner acting at least as a cushioning interface between the limb and socket. For the artificial limb to work in harmony with the user, the quality of the fit at this limb-socket interface is critical. A proper fit greatly reduces the risk of erosions, ulcers, or gangrene that could be created due to the tightness of the socket.

Use of an apparatus, device, and/or system described herein can provide a deeper understanding of how to effectively design and deliver prosthetic components to address the needs of patients and clinicians. Comfort is not solely achieved by equalization of pressures, it also includes the right circulation of air, right level of humidity, and the right alignment of the prosthesis. Moreover, building on current assessment techniques, the dynamic use of an apparatus described herein during typical amputee activities can provide a more accurate assessment process across a variety of measures. This novel approach can enable clinicians to have a deeper level of understanding of the in-socket interface dynamics during patient fitting; resulting in a more realistic view of what is occurring inside the socket while the patient is in motion. It is just as important to know how comfortable a socket is when the patient is wearing it in a static position, as it is when the patient is walking.

The use of an apparatus described herein leads to the alleviation of pain together with the facilitation of gains for amputees, prosthetists, and other users. This can be achieved by providing a device able to quantify socket comfort in static and dynamic conditions. Thereby, to improve the global socket design that will enable amputees to fully return to their daily activities and to have a higher quality of life.

The disclosed apparatus and related systems can enable prosthetic clinicians and users to achieve the best possible prosthetic socket fit for limb amputees. The apparatus/device can assess fit for new and experienced amputees during the fitting process based on a set of criteria. These criteria include: assessment of pressure distribution inside the socket, assessment of temperature levels, and alignment of the prosthesis system among others. The device/apparatus is capable of assessing quality of fit both statically and dynamically. It is intended for clinical, research, and home use. The device/apparatus is capable of tracking any changes on the residual limb or at the limb/socket interface that can affect the fit of the prosthesis over a certain period of time. The device/apparatus can be sued periodically over a period of days, weeks, months or years to monitor the fit as well as alternation so the fit over time. The device/apparatus is capable of collecting data and building a history of prosthesis's use that can be available for clinicians at any time.

The device/apparatus is generally composed of a sensor array embedded in a matrix. The matrix can be a gel like substance, such as silicone. The gel like substance can be other materials such as, but not limited to thermoplastic elastomers, polyurethanes, silicones, etc. (e.g., Spenco, Poron, Pelite, Plastazote, Nickelplast, Bock lite, pedilin and the like). The device/apparatus is configured to be pulled over or wrapped around a residual limb, much like a socket liner. In certain aspects the apparatus will have a proximal and distal end, with the proximal end being open for insertion of the residual limb and the distal being closed to cover the distal end of the residual limb and provide an interface with the socket. The device is easy to clean and data can be collected and transmitted to a connected computer or other device for processing and interpretation.

Data visualization can include options for LED's that change color with a proportional change of input to the sensor both on the device as well as a simulation on a connected computer. The device can work independently or in conjunction with software on a computer such as a server, a smartphone, a tablet, a desktop computer, and/or a laptop computer. Thus, a data visualization mechanism can be provided that changes color with respect to a proportional change of input to the sensor array. The data visualization mechanism can include a plurality of light emitting diodes that changes the color with respect to the proportional change of input to the sensor array. The data visualization mechanism can further include a computer simulation performed by the computing device with respect to the proportional change of input to the sensor array. The data visualization mechanism can provide visual results (e.g., depiction of the device and/or prosthesis) and show pressure points or alert flags to the prosthetist. This would be an easy and efficient manner to show results as well as an overall objective comfort measurement. Thus, prosthetist would be able to perform the appropriate changes to the socket aiming for the best prosthetic socket fit for the patient.

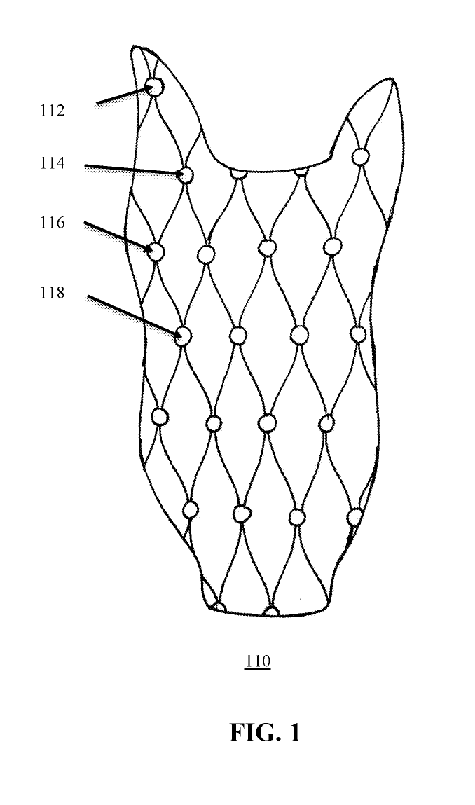

FIG. 1 illustrates a schematic diagram of an apparatus 110 for facilitating the best possible prosthetic fit in a prosthesis system for an amputee, in accordance with an example embodiment. Apparatus 110, which can also be referred to as an aperture comfort sensor or, for example, a prosthetic socket fit sensor can be implemented to alleviate pain (as discussed previously in the background section herein) by providing a tool that can be used in the clinic, or at home, and which measures several comfort indicators, enabling clinicians of any experience level to better assess and fit prosthetic sockets. Apparatus 110 has a proximal end configured to receive a residual limb and a distal end that cover the tip of residual limb.

Apparatus 110 is an assessment tool that can assist service providers in the prosthetic industry to provide a more reliable, comfortable, and high-quality end product to amputees. This can be achieved by providing a device that quantifies socket comfort and, thereby, improves socket designs that enable amputees to return more fully to their daily activities and have a higher quality of life. Apparatus 110 can also assist in tracking the use of the patient's artificial limb and to provide relevant information in regard to the current state of the residual limb and changes through a certain period of time.

Apparatus 110 includes a sensor array embedded in urethane or silicone similar to some prosthetic liners. The sensor array can be composed of a plurality of sensors such as, for example, sensors 112, 114, 116, 118 and so on. To measure the variety of comfort indicators required, there are several types of sensors that can be included in the context of such a sensor array. Examples of such sensors include pressure sensors, humidity and temperature sensors, and also alignment sensors (e.g., accelerometers). The sensors can be arranged in a mesh where all the sensors are evenly distributed within the socket or customized to increase the mesh density in user specified locations. In certain aspects a sensors 112, 114, 116, 118, and so can be configured with multiple sensors at each sensor point in the mesh or sensor lattice. The sensors or sensor points in the mesh can be positioned in the mesh with approximately 2, 4, 6, 8 to 10, 12, 14, 16 cm between sensors or sensor points, including all values and ranges there between.

Arrangement of wires between sensor nodes is designed to allow for expansion and contraction around the unique contorts of a user's residual limb. A preliminary representation of how the sensors are distributed inside the socket is depicted in FIG. 1. Apparatus 110 can measure not only the pressure at each of the pressure sensors embedded in the liner, but can also compare all pressure measurements in order to map in-socket pressures during use. Such information/data allows clinicians to better understand how a socket is fitting and identify adjustment areas. Similar mappings can be created for each of the comfort indicators measured. This sensor mesh can be used for both static (e.g., while the user is standing) and dynamic (e.g., while the user is walking) applications.

In order to obtain a comfortable socket, the pressure inside the socket should be properly distributed during both static and dynamic use. Because the total surface weight bearing socket designs are rarely successful due to the fluctuations of the residual limb throughout the day, specific weight bearing over select areas, such as the Patella Tendon, have been incorporated into socket designs in order to create a more practical and successful fitting. Appropriate loading on these areas, therefore, can be monitored using apparatus 110.

Sensors that can be used in accordance with one or more embodiments include, but are not limited to a pressure sensor, a humidity sensor, a temperature sensor (e.g., a Sensirion SHT21 Humidity and Temperature Sensor, and/or analog devices (e.g., ADXL3335 Accelerometer). A sensor used with apparatus 110 can be similar to, for example, the FlexiForce A301 sensor form Tekscan, Inc. (Boston, USA). In certain aspects the pressure sensor can be configured with a greater range of pressure measurement. In certain aspects pressure is from 0, 25, 50, 75, 100, 125, 150, 175, 200 to 225, 250, 275, 300, 325, 350, 375, 400 kPa, including all values and ranges there between.

The levels of temperature and humidity are important factors in prosthetic comfort. The resulting elevated skin temperatures may lead to discomfort, excess perspiration and promote skin injuries. Temperature in the residual limb inside a socket can vary from, for example, 25, 26, 27, 28 to 29, 30, 31, 32° C., including all values and ranges there between, depending on the ambient temperature, and relative humidity (RH) percentage ranges around, for example, 13, 14, 15, 16, 17, 18, 19, 20, 21, 22, 23, 24, 25, to 26, 27, 28, 29, 30, 31, 32, 33, 34, 35, 36, 37, 38, 39, 40, 41, 42, 43, 44, 45, 46, 47% RH, including all values and ranges there between. A Sensirion (Zurich, Switzerland) sensor, for example, offers a humidity and temperature sensor (e.g., model SHT21) that meets the application requirements of most common ranges of humidity in prosthetic sockets. This sensor is a 3×3×1.1 mm with a low power supply operation of 2.1-3.6 V maximum and works under −40 to 125° C. conditions. The relative humidity parameters of the sensor are: an accuracy of ±2% RH, an operating range of 0 to 100% RH, a response time of 8 s. The temperature parameters are an accuracy of ±0.3° C., an operating range of −40 to 125° C., and a response time of 5-30 s (Sensirion AG Switzerland, 2016). It can be appreciated, of course, that the aforementioned Sensrion sensor is discussed herein for purposes of example only and is not considered a limiting feature of the disclosed embodiments.

As part of a Micro-Electro-Mechanical Systems (MEMS), an accelerometer can be utilized to sense alignment and determine if the socket, or another modular prosthetic component, is not aligned properly. It can also be used to check for symmetry during user gait analysis. An example of an accelerometer that can be selected for use with apparatus 110 is the ADXL335 from Analog Devices (Norwood, Mass.), which has an accuracy of ±3 g and senses in 3-axes. This sensor offers a 4×4×1.45 mm component with a low power supply operation of 1.8 to 3.6 V. Again, it should be appreciated that the aforementioned ADXL335 device is one example of an accelerometer that can be used with apparatus 110 and that other possible accelerometer devices can be utilized instead. Such an accelerometer is not considered a limiting feature of the disclosed embodiments but is referred to for purposes of example only.

Devices described herein can include a microcontroller. In one example, all data collected from the sensors can be gathered and processed utilizing a data collection and transmission mechanism. As utilized herein the term “microcontroller” (also referred to as MCU, short for microcontroller unit) refers to a small computer (SoC) on a single integrated circuit containing a processor core, memory and programmable input/output peripherals. Program memory in the form of ferroelectric RAM, NOR flash or OTP ROM (Read only memory) may also be included on the chip, as well as a small amount of RAM (Random Access Memory). One example of such a microcontroller is the ATMEGA328P microcontroller from ATMEL The microcontroller can be used with a microcontroller board at the interface between the aforementioned sensor array of apparatus 110 and a microcontroller programed initially in, for example, LabVIEW (National Instruments, Austin, Tex.). It should be appreciated that the reference to, for example, the ATMEGA328P microcontroller and also LabVIEW is provided for purposes of example only and is not considered a limitation of the disclosed embodiments. An integrated circuit such as, for example, MAX2323, can be employed with apparatus 110 to connect the microcontroller with the computer and software to allow easy implementation of this device in a typical prosthetic clinic.

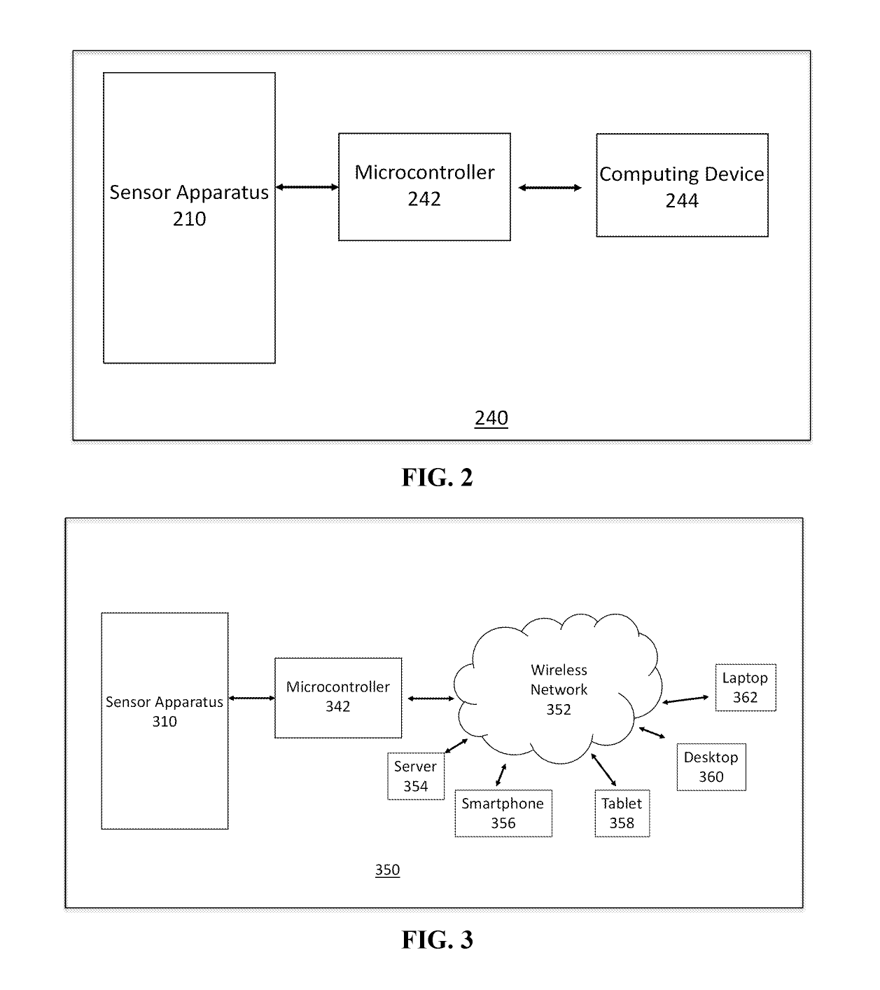

FIG. 2 illustrates an example of a system (240) for facilitating the best possible prosthetic fit in a prosthesis system for an amputee. Apparatus 210 can communicate with a microcontroller 242 (e.g., the microcontroller discussed above), which in turn can communicate with a computing device 244, which can be, for example, a computing device such as desktop computer, a server, a smartphone, a laptop computer, a smartwatch and so on. Communications between the microcontroller 242 can be implemented via, for example, direct wired connections (e.g., USB, Ethernet connections, etc.) or via wireless communications including, for example, cellular telecommunications, WiFi wireless communications, and Bluetooth wireless communications.

FIG. 3 illustrates an example of a system 350 for facilitating the best possible prosthetic fit in a prosthesis system for an amputee. In system 350, apparatus 310 and its sensor array can communicate with the microcontroller 342, which in turn communicates bidirectionally and wirelessly with a wireless network 352. Note that the wireless network 352 can be implemented as one or more packet based wireless networks, examples of which include a WiFi (e.g., 802.11 wireless network) and a cellular telecommunications network. A variety of computing devices can communicate with the wireless network 352 such as, for example, a server 354, a smartphone 356, a tablet computing device 358, a desktop computer 360, and/or a laptop computer 362.

Additional embodiments can thus be implemented, which include the use of a wireless system that pairs the aforementioned sensor array to, for example, a user's smartphone or tablet computing device and it's respective application for the phone. In some example embodiments, a custom circuit board may also be used.

The particular values and configurations discussed in these non-limiting examples can be varied and are cited to illustrate at least one embodiment and are not intended to limit the scope of the invention.

It will be appreciated that variations of the disclosed features and functions, or alternatives thereof, may be desirably combined into many other different systems or applications. It will also be appreciated that various presently unforeseen or unanticipated alternatives, modifications, variations or improvements therein may be subsequently made by those skilled in the art, which are also intended to be encompassed by the following claims.

What is claimed is:

1. An apparatus for achieving a best possible prosthetic fit in a prosthesis system for an amputee, said apparatus comprising:
a sleeve or sock configured to cover a residual limb of the amputee, the sleeve or sock having an embedded sensor array; and
a data collection and transmission component that is in communication with the sensor array and configured to transmit data from the sensor array to a computing device for processing and interpretation in order to assess a quality of fit of a prosthesis into which the residual limb is placed.

2. The apparatus of claim 1, wherein the sensor array is embedded in a gel matrix.

3. The apparatus of claim 2, wherein the gel matrix is a silicon matrix.

4. The apparatus of claim 1 wherein the sensor array is arranged as a sensor mesh.

5. The apparatus of claim 1 wherein the sensor array includes at least one pressure sensor, at least one temperature sensor, at least one accelerometer or any combination thereof.

6. The apparatus of claim 1 wherein the data is processed and interpreted by a computing device to generate a map of pressure distribution on the contours of the residual limb when engaged with a prosthesis socket.

7. The apparatus of claim 1 wherein the data is processed and interpreted by said computing device to generate a map of residual limb temperature levels when engaged with a prosthesis socket.

8. The apparatus of claim 1 wherein said data is processed and interpreted by said computing device to derive alignment data associated with the residual limb and a prosthesis.

9. The apparatus of claim 1 further comprising a data visualization display that changes color with respect to changes in data received from the sensor array.

10. The apparatus of claim 9 wherein the data visualization display comprises a plurality of light emitting diodes that change color with respect to a change in data received from the sensor array.

11. The apparatus of claim 9 wherein the data visualization display is configured to present to a user a computer simulation generated by the computing device, the computer simulation illustrating the data received from the sensor array.

12. The apparatus of claim 1 wherein the data collection and transmission mechanism comprises a microcontroller in communication with the sensors.
